(19) United States
(12) Patent Application Publication
(10) Pub. No.: US 2020/0369982 A1
(43) Pub. Date: Nov. 26, 2020

(54) PROCESS FOR PRODUCTION OF CONCENTRATED POLYUNSATURATED FATTY ACID OILS

(71) Applicant: DSM IP Assets B.V., Heerlen (NL)

(72) Inventors: Jaroslav KRALOVEC, Halifax (CA); Erick REYES-SUAREZ, Dartmouth (CA)

(21) Appl. No.: 16/636,938

(22) PCT Filed: Aug. 6, 2018
(86) PCT No.: PCT/EP2018/071213

§ 371 Date: Feb. 6, 2020

Related U.S. Application Data
(60) Provisional application No. 62/542,053, filed on Aug. 7, 2017.

(51) Int. Cl. C11C 3/00; A23L 33/00; A23L 33/115; A23L 2/52; C11C 1/00; C11C 1/10; C12P 7/64

(52) U.S. Cl. CPC C11C 3/003; A23L 33/40; A23L 33/115; C12P 7/6427; C11C 1/005; C11C 1/10; C11C 1/002; A23L 2/52

(57) ABSTRACT

The present invention relates to oil compositions that are enriched in polyunsaturated fatty acids; compositions containing the oil compositions; and methods of making and using the oil compositions. The oil is preferably a microbial or marine oil.

### CROSS-REFERENCE TO RELATED APPLICATIONS

This application claims the benefit of the filing date of U.S. Provisional Patent Application No. 62/542,053 filed Aug. 7, 2017, the disclosure of which is hereby incorporated herein by reference.

### FIELD OF THE INVENTION

The present disclosure relates to oil compositions that are enriched in polyunsaturated fatty acids, particularly arachidonic acid; compositions containing the oil compositions; and methods of making and using the oil compositions.

### BACKGROUND OF THE INVENTION

Fatty acids are classified based on the length and saturation characteristics of the carbon chain. Fatty acids are termed short chain, medium chain, or long chain fatty acids based on the number of carbons present in the chain, are termed saturated fatty acids when no double or triple bonds are present between the carbon atoms, and are termed unsaturated fatty acids when double or triple bonds are present. Unsaturated long chain fatty acids are monounsaturated when only one double or triple bond is present and are polyunsaturated when more than one double or triple bond is present.

Polyunsaturated fatty acids (PUFAs) are classified based on the position of the first double bond from the methyl end of the fatty acid; omega-3 (n-3) fatty acids contain a first double bond at the third carbon counting from the methyl terminal, while omega-6 (n-6) fatty acids contain a first double bond at the sixth carbon. For example, docosahexaenoic acid (“DHA”) is an omega-3 long chain polyunsaturated fatty acid (LC-PUFA) with a chain length of 22 carbons and 6 double bonds, often designated as “22:6 n-3.” Other omega-3 LC-PUFAs include eicosapentaenoic acid (“EPA”), designated as “20:5 n-3,” and omega-3 docosapentaenoic acid (“DPA n-3”), designated as “22:5 n-3.” Omega-6 LC-PUFAs include arachidonic acid (“ARA”), designated as “20:4 n-6,” and omega-6 docosapentaenoic acid (“DPA n-6”), designated as “22:5 n-6.”

Arachidonic acid (ARA, 20:4 n-6) is an LC-PUFA belonging to the omega-6 category. This molecule can undergo either monooxygenation or epoxidation by enzymes in the cytochrome P450 (CYP450) family and the metabolites have different biological functions based on sites of production—the endothelium of vessels in many organs, the lungs, the tubular and corneal epithelium, the liver, etc. The likely targets are either enzymes (Na+-K+-ATPase) or ion channels (calcium activated potassium channels). Skeletal muscle is an especially active site of arachidonic acid retention. In addition to being involved in cellular signalling as a lipid second messenger involved in the regulation of signalling enzymes, such as PLC-γ, PLC-δ, and PKC-α, -β, and -γ isoforms, arachidonic acid is a key inflammatory intermediate and acts as a vasodilator.

ARA-concentrated oils could be used alone as a building block for specialized active pharmaceutical ingredient (API) molecules, e.g., those directed to the management of pain, inflammation, neurological and brain diseases, and cognitive impairment. It could also be used as a dietary supplement alone, in combination with omega-3, omega-7 and other suitable substances for prevention or management of pain and inflammation, neurological and brain diseases, and cognitive impairment. Access to this ARA concentrate opens additional possibilities, e.g., manufacturing of other highly potent ARA forms.

Methods for concentrating omega-3 fatty acids such as EPA and DHA from starting oils are known and are relatively straightforward, resulting in greater than 95% potency. However, concentrating ARA provides a significantly greater challenge, generally due to the composition of the starting oil that typically is obtained from fermentation of the fungus from the genus Mortierella.

Thus, there remains a need for methods to concentrate ARA from a starting oil to obtain high potency ARA oils in a respectable yield.

The solution to this technical problem is provided by the embodiments characterized in the claims.

### SUMMARY OF THE INVENTION

The present application relates to a process for the production of ARA-rich oil from a starting oil, e.g., an oil obtained from fermentation of Mortierella alpina. Generally, the process comprises transesterification of a starting oil to its corresponding ethyl ester form, e.g., by use of dry ethanol in the presence of sodium ethoxide, followed by distillation, e.g., wiped-film evaporation, fractional distillation, or short path distillation. The distillate is then subjected to a first urea complexation step using, e.g., urea in 95% ethanol. After the isolation of the intermediate, the intermediate is further fractionated by reverse phase chromatography in methanol/water under isocratic conditions. The appropriate fractions are collected, concentrated, and subjected to a second urea complexation step, with the product isolated and stabilized.

The present application also discloses oils produced by the process described herein.

The present application further discloses microbial oils comprising at least about 70% by weight ARA. In particular, the microbial oil may be obtained from one or more microorganisms, such as, e.g., microalgae, bacteria, fungi, and protists. In some embodiments, the microorganism is a fungus. In a preferred embodiment, the fungus is of the genus Mortierella. In a more preferred embodiment, the microorganism is of the species Mortierella alpina.

Also provided are food products, cosmetics, or pharmaceutical compositions for a non-human or human, comprising the oils described herein. In some embodiments, the food product is a milk, a beverage, a therapeutic drink, a nutritional drink, or a combination thereof. In a preferred embodiment, the food product is an infant formula or a dietary supplement.

### BRIEF DESCRIPTION OF THE DRAWINGS

For a further understanding of the nature, objects, and advantages of the present disclosure, reference should be had to the following detailed description, read in conjunction with the following drawings, wherein like reference numerals denote like elements.

FIG. 1 shows an exemplary process of the invention.

FIG. 2 shows a comparative process.

### DETAILED DESCRIPTION OF THE INVENTION

Before the subject disclosure is further described, it is to be understood that the disclosure is not limited to the particular embodiments of the disclosure described below, as variations of the particular embodiments may be made and still fall within the scope of the appended claims. It is also to be understood that the terminology employed is for the purpose of describing particular embodiments, and is not intended to be limiting. Instead, the scope of the present disclosure will be established by the appended claims.

In this specification and the appended claims, the singular forms “a,” “an,” and “the” include plural reference unless the context clearly dictates otherwise. Unless defined otherwise, all technical and scientific terms used herein have the same meaning as commonly understood to one of ordinary skill in the art to which this disclosure belongs.

The subject disclosure features, in one aspect, a process for the production of a long chain-polyunsaturated fatty acid (LC-PUFA)-rich oil. In a preferred embodiment, the LC-PUFA is an omega-6 fatty acid. In a more preferred embodiment, the LC-PUFA is arachidonic acid (ARA).

Starting Oil Composition

In some embodiments, the starting oil is a microbial or marine oil.

Oil produced by a microorganism or obtained from a microbial cell is referred to as “microbial oil”. Oil produced by algae and/or fungi is referred to as an algal and/or a fungal oil, respectively.

As used herein, a “microorganism” refers to organisms such as algae, bacteria, fungi, protist, yeast, and combinations thereof, e.g., unicellular organisms. A microorganism includes but is not limited to, golden algae (e.g., microorganisms of the kingdom Stramenopiles); green algae; diatoms; dinoflagellates (e.g., microorganisms of the order Dinophyceae including members of the genus Crypthecodinium such as, for example, Crypthecodinium cohnii or C. cohnii); microalgae of the order Thraustochytriales; yeast (Ascomycetes or Basidiomycetes); and fungi of the genera Mucor, Mortierella, including but not limited to Mortierella alpina and Mortierella sect. schmuckeri, and Pythium, including but not limited to Pythium insidiosum.

In one embodiment, the microorganisms are from the genus Mortierella, genus Crypthecodinium, genus Thraustochytrium, and mixtures thereof. In a further embodiment, the microorganisms are from Crypthecodinium Cohnii. In a further embodiment, the microorganisms are from Mortierella alpina. In a still further embodiment, the microorganisms are from Schizochytrium sp. In yet an even further embodiment, the microorganisms are selected from Crypthecodinium Cohnii, Mortierella alpina, Schizochytrium sp., and mixtures thereof.

In a still further embodiment, the microorganisms include, but are not limited to, microorganisms belonging to the genus Mortierella, genus Conidiobolus, genus Pythium, genus Phytophthora, genus Penicillium, genus Cladosporium, genus Mucor, genus Fusarium, genus Aspergillus, genus Rhodotorula, genus Entomophthora, genus Echinosporangium, and genus Saprolegnia.

In an even further embodiment, the microorganisms are from microalgae of the order Thraustochytriales, which includes, but is not limited to, the genera Thraustochytrium (species include arudimentale, aureum, benthicola, globosum, kinnei, motivum, multirudimentale, pachydermum, proliferum, roseum, striatum); the genera Schizochytrium (species include aggregatum, limnaceum, mangrovei, minutum, octosporum); the genera Ulkenia (species include amoeboidea, kerguelensis, minuta, profunda, radiate, sailens, sarkariana, schizochytrops, visurgensis, yorkensis); the genera Aurantiacochytrium; the genera Oblongichytrium; the genera Sicyoidochytium; the genera Parientichytrium; the genera Botryochytrium; and combinations thereof. Species described within Ulkenia will be considered to be members of the genus Schizochytrium. In another embodiment, the microorganisms are from the order Thraustochytriales. In yet another embodiment, the microorganisms are from Thraustochytrium. In still a further embodiment, the microorganisms are from Schizochytrium sp.

In certain embodiments, the oil can comprise a marine oil. Examples of suitable marine oils include, but are not limited to, Atlantic fish oil, Pacific fish oil, or Mediterranean fish oil, or any mixture or combination thereof. In more specific examples, a suitable fish oil can be, but is not limited to, pollack oil, bonito oil, pilchard oil, tilapia oil, tuna oil, sea bass oil, halibut oil, spearfish oil, barracuda oil, cod oil, menhaden oil, sardine oil, anchovy oil, capelin oil, herring oil, mackerel oil, salmonid oil, tuna oil, and shark oil, including any mixture or combination thereof. Other marine oils suitable for use herein include, but are not limited to, squid oil, cuttle fish oil, octopus oil, krill oil, seal oil, whale oil, and the like, including any mixture or combination thereof.

Esterification

In some embodiments, a fatty acid as described herein can be a fatty acid ester or ester. In some embodiments, a fatty acid ester includes an ester of an omega-3 fatty acid, omega-6 fatty acid, and combinations thereof. In some embodiments, the fatty acid ester is an ARA ester. In some embodiments, an oil or fraction thereof as described herein is esterified to produce an oil or fraction thereof comprising fatty acid esters. The term “ester” refers to the replacement of the hydrogen in the carboxylic acid group of the fatty acid molecule with another substituent. Examples of esters include methyl, ethyl, propyl, butyl, pentyl, t-butyl, benzyl, nitrobenzyl, methoxybenzyl, benzhydryl, and trichloro ethyl. In some embodiments, the ester is a carboxylic acid protective ester group, esters with aralkyl (e.g., benzyl, phenethyl), esters with lower alkenyl (e.g., allyl, 2-butenyl), esters with lower-alkoxy-lower-alkyl (e.g., methoxymethyl, 2-methoxyethyl, 2-ethoxyethyl), esters with lower-alkanoyloxy-lower-alkyl (e.g., acetoxymethyl, pivaloyloxymethyl, 1-pivaloyloxyethyl), esters with lower-alkoxycarbonyl-lower-alkyl (e.g., methoxycarbonylmethyl, isopropoxycarbonylmethyl), esters with carboxy-lower alkyl (e.g., carboxymethyl), esters with lower-alkoxycarbonyloxy-lower-alkyl (e.g., 1-(ethoxycarbonyloxy)ethyl, 1-(cyclohexyloxycarbonyloxy)ethyl), esters with carbamoyloxy-lower alkyl (e.g., carbamoyloxymethyl), and the like. In some embodiments, the added substituent is a linear or cyclic hydrocarbon group, e.g., a C1-C6 alkyl, C1-C6 cycloalkyl, C1-C6 alkenyl, or C1-C6 aryl ester. In some embodiments, the ester is an alkyl ester, e.g., a methyl ester, ethyl ester or propyl ester. In some embodiments, the ester substituent is added to the free fatty acid molecule when the fatty acid is in a purified or semi-purified state.

Fatty acid esters, in particular polyunsaturated fatty acid esters, can be made in ways that are known to one of ordinary skill in the art.

For example, tri-acyl glycerides, di-acyl glycerides, and/or mono-acyl glycerides that contain fatty acids, particularly polyunsaturated fatty acids, can be reacted with an alcohol in the presence of an acid or a base to produce esters. The disclosure of U.S. Publication No. 2009/0023808 is incorporated by reference herein in its entirety.

The base can be, for example, a metal alkyloxide. Metal alkyloxides include sodium ethoxide, sodium methoxide, sodium n-propoxide, sodium iso-propoxide, sodium n-butoxide, sodium iso-butoxide, sodium sec-butoxide, sodium tert-butoxide, sodium n-pentoxide, sodium n-hexoxide, lithium ethoxide, lithium methoxide, lithium n-propoxide, lithium iso-propoxide, lithium n-butoxide, lithium iso-butoxide, lithium sec-butoxide, lithium tert-butoxide, lithium n-pentoxide, lithium n-hexoxide, potassium ethoxide, potassium methoxide, potassium n-propoxide, potassium iso-propoxide, potassium n-butoxide, potassium iso-butoxide, potassium sec-butoxide, potassium tert-butoxide, potassium n-pentoxide, and/or potassium n-hexoxide.

In some situations, the base can be made by adding sodium metal, potassium metal, or lithium metal to an alcoholic solution.

In some situations, the base can be made by adding a metal hydride, such as lithium hydride, sodium hydride, or potassium hydride, to an alcoholic solution.

The ratio of base to oil, on a weight to weight basis can, for example, range from 1:1 to 1000:1, including all values and subranges therebetween as if explicitly written out. For example, the ratio of base to oil, on a weight to weight basis, can be 2:1, 3:1, 4:1, 5:1, 6:1, 7:1, 8:1, 9:1, 10:1, 20:1, 30:1, 40:1, 50:1, 60:1, 70:1, 80:1, 90:1, 100:1, 200:1, 300:1, 400:1, 500:1, 600:1, 700:1, 800:1, or 900:1.

The esterification reaction can be run at a temperature ranging from 10° C. to 100° C., including all values and subranges therebetween as if explicitly written out. For example, the esterification reaction can be run at 20° C., 30° C., 40° C., 50° C., 60° C., 70° C., 80° C., or 90° C.

The esterification reaction can be run open to the atmosphere, or under an inert atmosphere such as nitrogen or argon.

Workup and isolation of the fatty acid esters can be done in ways known to one of ordinary skill, for example, by extraction with an organic solvent, water, or a supercritical fluid. The organic solvent can be, for example, pentane, hexane, di-ethyl ether, ethyl acetate, or a combination of these. The water can optionally contain other substances such as sodium bicarbonate, sodium carbonate, ammonium chloride and/or dilute mineral acid. The supercritical fluid can be, for example, carbon dioxide.

In some embodiments, the oil is sometimes transesterified back to convert at least part of the ester fraction in the oil to a triglyceride fraction. Transesterification, in particular transesterification of polyunsaturated fatty acid esters, can be made in ways that are known to one of ordinary skill in the art.

Distillation

In some embodiments, the process comprises subjecting the oil to at least one distillation step comprising feeding the esterified oil to at least one apparatus and subjecting the esterified oil to conditions to remove low-boiling compounds in a distillate.

The distillation step can be fractional distillation, short path distillation, falling-film evaporation, wiped-film evaporator, or a combination thereof. In a preferred embodiment, the distillation step is fractional distillation. In another preferred embodiment, the distillation step is short path distillation.

The distillation can be performed by any means known to those of ordinary skill in the art.

Urea Complexation

In some embodiments, the process comprises subjecting the oil to at least one urea complexation step. In a preferred embodiment, the process comprises at least two urea complexation steps. Urea complexation may be performed using any method known to those of skill in the art.

The term “urea/oil complex” is used synonymously herein with “urea adduct” or “clathrate.” The urea/oil complex can be produced in a commercial or laboratory oil processing step wherein oils from any of a variety of sources are contacted with urea. Urea preferentially forms a complex with saturated and monounsaturated fatty acids/esters in the oil and is called a urea/oil complex or urea adduct. Thus, the urea/oil complex is a composition containing urea and saturated and/or monounsaturated fatty acids/esters. While the remaining fraction of the oil is rich in PUFAs, some PUFAs can be complexed with the urea and become part of the urea/oil complex. Solvents are also used in this process and so residual solvent is often a part of the urea/oil complex. The disclosed methods thus begin with a urea/oil complex that comprises urea, saturated and monounsaturated fatty acids/esters that are associated with the urea, a residual amount of solvent, and optionally an undesirable residual amount of PUFAs.

The urea that can be used to form the urea/oil complex can be obtained from a variety of commercial sources. Examples of suitable sources for urea include Acros Organics (Morris Plains, N.J.), Fisher Scientific (Pittsburgh, Pa.), or Sigma Aldrich (St. Louis, Mo.).

The urea and oil can be combined in the presence of a solvent to form the urea/oil complex. Thus, as a result of the use of solvent in the production of the urea/oil complex, the complex can, and most often does, comprise residual amounts of solvent. In some embodiments, the solvent is an alcohol (e.g., ethanol). Preferably, the solvent is 190 proof ethanol (i.e., 95% ethanol).

In some embodiments, the urea/oil complex is prepared by dissolving urea in ethanol to form a urea/ethanol solution. The ratio of urea to ethanol in the reaction mixture can be from about 1:0.1 to about 1:10, more typically about 1:1.5. To facilitate dissolution of the urea in ethanol, the mixture can be heated. Suitable temperatures to which the ethanol and urea can be mixed include, but are not limited to, from about 60° C. to about 100° C., from about 65° C. to about 95° C., from about 70° C. to about 90° C., or from about 75° C. to about 85° C. For example, the mixture can be heated to from about 85° C. to about 90° C.

The oil can be combined with the urea/ethanol solution at an elevated temperature (i.e., a hot urea/ethanol solution) to form the complex. Optionally, the oil is degassed and/or heated prior to combining the oil with the hot urea/ethanol solution. In some examples, the oil is heated to a temperature within about 15° C. of the hot urea/ethanol solution. For example, when the urea/ethanol solution is at a temperature of about 85° C. to about 90° C., the oil can be heated to a temperature of about 80° C. prior to combining it with the urea/ethanol solution. The oil is mixed with the urea/ethanol solution and the combined mixture is allowed to cool to form the solid urea/oil complex. The same procedures can be used with other solvents.

The ratio of the urea to oil in the reaction mixture can be from about 0.1:1 to about 2:1, more typically about 0.5:1.5, about 0.85:1, or about 1.2:1. The urea/oil complex is then usually separated from the remaining oil, e.g., by filtration.

The disclosed methods include the step of taking the urea/oil complex (urea adduct) and removing the residual solvent (e.g., ethanol) to form a dried urea/oil complex (also referred to as a urea “cake”). The dried urea/oil complex is substantially free of solvent. By “substantially free of solvent” is meant that the dried urea/oil complex contains less than about 1 wt. %, less than about 0.5 wt. %, or less than about 0.1 wt. % solvent. The solvent can be removed under vacuum. Suitable temperatures for performing the solvent removal include, but are not limited to, from about 4° C. to about 60° C., preferably from about 10° C. to about 22° C. In other examples, the solvent can be removed at about 5° C., about 10° C., about 15° C., about 20° C., about 25° C., about 30° C., about 35° C., about 40° C., about 45° C., about 50° C., about 55° C., or about 60° C., where any of the stated values can form an upper and/or lower endpoint of a range.

After removing the solvent from the urea/oil complex, the dried urea/oil complex or cake is combined with water. The urea component of the dried urea/oil complex dissolves in the water. This dissolution of urea can be facilitated further at an elevated temperature due, in part, to the increased solubility of urea in water at elevated temperatures. The solubility of urea in water at ambient temperature is about 108 g of urea per 100 mL of water. However, at about 60° C. to about 80° C., the solubility of urea in water increases to about 250-400 grams of urea per 100 mL of water. Thus, in preferred embodiments, the water combining step is performed at temperatures that include, but are not limited to, from about 50° C. to about 80° C., from about 55° C. to about 75° C., or from about 60° C. to about 70° C. In some examples, the dried urea/oil complex can be combined with water at about 50° C., about 55° C., about 60° C., about 65° C., about 70° C., about 75° C., or about 80° C., where any of the stated values can form an upper and/or lower endpoint of a range. In some specific examples, the dried urea/oil complex can be combined with water at about 60° C. to about 80° C., or more specifically, from about 65° C. to about 75° C. or, still more specifically, at about 72° C. Optionally, the water is heated to the elevated temperature and provided to the dried urea/oil complex at the elevated temperature.

Due to the increased solubility of urea in water at elevated temperatures, a minimal amount of water can be used in this step to form an aqueous concentrated urea solution. The total amount of water added will of course depend on how much urea is present in the cake. In some embodiments, the water in the combining step is provided at about 30% by weight to about 50% by weight of the dried urea/oil complex. For example, water can be provided at about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, or about 50% by weight of the dried urea/oil complex, where any of the stated values can form an upper and/or lower endpoint of a range. In some examples, the water in the combining step is provided at about 40% by weight of the dried urea/oil complex.

It is also possible to perform this step repeatedly by, i.e., combining the dried urea/oil complex with water, separating the aqueous layers, and then combining the dried urea/oil complex with water again. Still further, this step can be performed under a nitrogen atmosphere with stirring.

As noted, combining the dried urea/oil complex with water forms two phases: an aqueous concentrated urea solution, containing the dissolved urea, and an organic phase, containing the oil (saturated and/or monosaturated fatty acids and optionally, PUFAs). The two phases can then be allowed to separate further into an aqueous layer and an organic layer. Phase separation can be performed at a temperature from about 50° C. to about 80° C. For example, the separation step can be performed at a temperature of from about 55° C. to about 75° C., or from about 60° C. to about 70° C. In some examples, the two phases can be allowed to separate at about 50° C., about 55° C., about 60° C., about 65° C., about 70° C., about 75° C., or about 80° C., where any of the stated values can form an upper and/or lower endpoint of a range.

Oil can be recovered from the organic phase, sometimes in significant amounts, by washing with water and drying the layer.

Concentration Methods

In some embodiments, the process comprises subjecting the oil to at least one concentration step. In some embodiments, the concentration step comprises chromatography, distillation, urea complexation, and combinations thereof.

In one embodiment, the concentration step comprises chromatography. In one preferred embodiment, the chromatography is silver ion chromatography. In a more preferred embodiment, the chromatography is simulated moving bed chromatography. In a further preferred embodiment, the chromatography is reverse phase chromatography.

In one embodiment, the concentration step comprises distillation. In one preferred embodiment, the distillation is fractional distillation. In another preferred embodiment, the distillation is short path distillation.

In one embodiment, the concentration step comprises urea complexation.

Reverse phase chromatography

In some embodiments, the process comprises subjecting the oil to at least one reverse phase chromatography step. Reverse phase chromatography may be performed using any method known to those of skill in the art.

In some embodiments, the chromatography column may have internal diameter of approximately 3.8 cm, an effective length of approximately 27 cm, and/or a volume of approximately 313 mL.

In some embodiments, the chromatography column is packed with a silica matrix. In a preferred embodiment, the silica matrix is a C18-bonded silica gel. In a preferred embodiment, the particle size of the matrix is from approximately 40 μm to approximately 63 μm.

In some embodiments, the eluent comprises methanol. In a preferred embodiment, the eluent is a mixture of methanol and water.

In some embodiments, the eluent comprises at least about 80% methanol, at least about 85% methanol by weight, at least about 90% methanol by weight, or at least about 95% methanol by weight. In some embodiments, the eluent comprises at least 80% methanol by weight, at least 85% methanol by weight, at least 90% methanol by weight, or at least 95% methanol by weight.

In some embodiments, the eluent comprises less than about 20% water by weight, less than about 15% water by weight, less than about 10% water by weight, or less than about 5% water by weight. In some embodiments, the eluent comprises less than 20% water by weight, less than 15% water by weight, less than 10% water by weight, or less than 5% water by weight.

In some embodiments, the eluent comprises from about 80% to about 95% methanol by weight. In a preferred embodiment, the eluent comprises from about 90% to about 95% methanol by weight. In a more preferred embodiment, the eluent comprises about 92% methanol by weight.

In some embodiments, the eluent comprises from about 5% to about 20% water by weight. In a preferred embodiment, the eluent comprises from about 5% to about 10% water by weight. In a more preferred embodiment, the eluent comprises about 8% water by weight.

In some embodiments, the oil is eluted from the chromatography column in isocratic mode.

Column Chromatography

In some embodiments, the process comprises column chromatography. A column is packed with a stationary phase such as silica gel, alumina, and/or silica gel impregnated with silver nitrate, and wetted with a solvent or a mixture of solvents. The oil containing the desired product(s) is loaded onto the column, and eluted with one or more mobile phase solvents such as dimethyl sulfoxide, N, N-dimethyl formamide, ethyl acetate, hexane, methanol, acetone, ethanol, propanol, tetrahydrofuran, diethyl ether, pentane, dichloromethane, chloroform, and tetrahydropyran.

The desired product(s) can be collected in fractions (e.g., in test tubes) and then can be concentrated by solvent removal, under reduced pressure, or alternatively by blowing an inert atmosphere over the collected factions, to yield an oil enriched in the desired product(s).

Fractional Distillation

In some embodiments, the process comprises fractional distillation. The oil is placed in a heating flask, and the flask is heated, optionally under reduced pressure. In one example, the desired product(s) convert to vapor phase when their boiling points are reached and pass through a fractionating column, are condensed in a condenser, and collected in a receiving flask. In an alternative example, the desired product(s) stay in the heating flask and impurities distill away from the desired product(s).

The fractional distillation can be run at a temperature ranging from, for example, 40° C. to 500° C., including all values and subranges therebetween as if explicitly written out. For example, the fractional distillation can be run at 50° C., 60° C., 70° C., 80° C., 90° C., 100° C., 110° C., or 120° C., 130° C., 140° C., 150° C., 160° C., 170° C., 180° C., 190° C., 200° C., 210° C., 220° C., 230° C., or 240° C., 250° C., 260° C., 270° C., 280° C., 290° C., 300° C., 310° C., 320° C., 330° C., 340° C., 350° C., or 360° C., 370° C., 380° C., 390° C., 400° C., 410° C., 420° C., 430° C., 440° C., 450° C., 460° C., 470° C., 480° C., or 490° C.

The fractional distillation is preferably conducted under reduced pressure. The reduced pressure can range from 0.0001 atmospheres to 0.9 atmospheres, including all values and subranges therebetween as if explicitly written out. An atmosphere is abbreviated “atm” and is equivalent to 101,325 Pa. The reduced pressure can be, for example, 0.001 atm, 0.01 atm, 0.1 atm, 0.2 atm, 0.3 atm, 0.4 atm, 0.5 atm, 0.6 atm, 0.7 atm, or 0.8 atm.

Solid Phase Extraction

In some embodiments, the process comprises subjecting the oil to at least one solid phase extraction step. Solid phase extraction (SPE) may be performed using any method known to those of skill in the art.

In some embodiments, the SPE step is performed using a silica SPE cartridge.

In the present invention, any concentrating, reacting, and/or purifying technique can be combined with any other concentrating, reacting, and/or purifying technique to produce microbial oils enriched in: polyunsaturated fatty acids, their esters, their salts, aldehydes thereof and/or alcohols thereof. The enrichment techniques can be used in any order and combination.

Resulting Oil Composition

In some embodiments, the oil comprises one or more LC-PUFAs. In some embodiments, the oil comprises at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% LC-PUFA. In a preferred embodiment, the LC-PUFA is in ester form. In a more preferred embodiment, the ester is an ethyl ester. In a preferred embodiment, the % by weight of the LC-PUFA is the % by weight of the oil. In a more preferred embodiment, the % by weight of the LC-PUFA is the % by weight of the fatty acids in an ester fraction. In some embodiments, the LC-PUFA is in triglyceride form.

In one embodiment, the oil comprises at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99% by weight ARA. In a preferred embodiment, the ARA is in ester form. In a more preferred embodiment, the ester is an ethyl ester. In a preferred embodiment, the % by weight of ARA is the % by weight of the oil. In a more preferred embodiment, the % by weight of the ARA is the % by weight of the fatty acids in an ester fraction.

In some embodiments, the oil comprises about 3% to about 13%, about 4% to about 12%, about 5% to about 11%, about 6% to about 10%, or about 7% to about 9% linoleic acid (“LA”). In a preferred embodiment, the LA is in ester form. In a more preferred embodiment, the ester is an ethyl ester. In a preferred embodiment, the % by weight of the LA is the % by weight of the oil. In a more preferred embodiment, the % by weight of the LA is the % by weight of the fatty acids in the ester fraction.

In some embodiments, the oil comprises from about 0.5% to about 5%, about 1% to about 5%, or about 3% to about 4% LA. In a preferred embodiment, the LA is in ester form. In a more preferred embodiment, the ester is an ethyl ester. In a preferred embodiment, the % by weight of the LA is the % by weight of the oil. In a more preferred embodiment, the % by weight of the LA is the % by weight of the fatty acids in an ester fraction.

In some embodiments, the oil comprises less than about 5%, less than about 4%, less than about 3%, less than about 2.5%, less than about 2%, less than about 2%, less than about 1.5%, less than about 1%, or less than about 0.5% EPA. In a preferred embodiment, the EPA is in ester form. In a more preferred embodiment, the ester is an ethyl ester. In a preferred embodiment, the % by weight of the EPA is the % by weight of the oil. In a more preferred embodiment, the % by weight of the EPA is the % by weight of the fatty acids in an ester fraction.

In some embodiments, the oil comprises from about 0.1% to about 5%, about 0.5% to about 3%, or about 1% to about 2% EPA. In a preferred embodiment, the EPA is in ester form. In a more preferred embodiment, the ester is an ethyl ester. In a preferred embodiment, the % by weight of the EPA is the % by weight of the oil. In a more preferred embodiment, the % by weight of the EPA is the % by weight of the fatty acids in an ester fraction.

In some embodiments, the oil comprises less than about 5%, less than about 4%, less than about 3%, less than about 2.5%, less than about 2%, less than about 2%, less than about 1.5%, less than about 1%, or less than about 0.5% DHA. In a preferred embodiment, the DHA is in ester form. In a more preferred embodiment, the ester is an ethyl ester. In a preferred embodiment, the % by weight of the DHA is the % by weight of the oil. In a more preferred embodiment, the % by weight of the DHA is the % by weight of the fatty acids in an ester fraction.

In some embodiments, the oil comprises from about 0.1% to about 5%, about 0.5% to about 3%, or about 1% to about 2% DHA. In a preferred embodiment, the DHA is in ester form. In a more preferred embodiment, the ester is an ethyl ester. In a preferred embodiment, the % by weight of the DHA is the % by weight of the oil. In a more preferred embodiment, the % by weight of the DHA is the % by weight of the fatty acids in an ester fraction

In some embodiments, the oil comprises less than about 5%, less than about 4%, less than about 3%, less than about 2.5%, less than about 2%, less than about 2%, less than about 1.5%, less than about 1%, or less than about 0.5% gamma-linolenic acid (“GLA”). In a preferred embodiment, the GLA is in ester form. In a more preferred embodiment, the ester is an ethyl ester. In a preferred embodiment, the % by weight of the GLA is the % by weight of the oil. In a more preferred embodiment, the % by weight of the GLA is the % by weight of the fatty acids in an ester fraction.

In some embodiments, the oil comprises less than about 5%, less than about 4%, less than about 3%, less than about 2.5%, less than about 2%, less than about 2%, less than about 1.5%, less than about 1%, or less than about 0.5% dihomo-gamma-linolenic acid (“DGLA”). In a preferred embodiment, the DGLA is in ester form. In a more preferred embodiment, the ester is an ethyl ester. In a preferred embodiment, the % by weight of the DGLA is the % by weight of the oil. In a more preferred embodiment, the % by weight of the DGLA is the % by weight of the fatty acids in an ester fraction.

In some embodiments, the oil comprises less than about 5%, less than about 4%, less than about 3%, less than about 2.5%, less than about 2%, less than about 2%, less than about 1.5%, less than about 1%, or less than about 0.5% stearidonic acid (“SDA”). In a preferred embodiment, the SDA is in ester form. In a more preferred embodiment, the ester is an ethyl ester. In a preferred embodiment, the % by weight of the SDA is the % by weight of the oil. In a preferred embodiment, the % by weight of the SDA is the % by weight of the fatty acids in an ester fraction.

In some embodiments, the comprises less than about 5%, less than about 4%, less than about 3%, less than about 2.5%, less than about 2%, less than about 2%, less than about 1.5%, less than about 1%, or less than about 0.5% of a polyunsaturated fatty acid having greater than 22 carbons (very long chain PUFAs). In some embodiments, the very long chain PUFA is 7,10,13,16,19,22,25 octacosaoctaenoic acid (C28:8). In a preferred embodiment, the oil comprises 0% 7,10,13,16,19,22,25 octacosaoctaenoic acid (C28:8). In a preferred embodiment, the very long chain PUFA is in ester form. In a more preferred embodiment, the ester is an ethyl ester. In a preferred embodiment, the % by weight of the very long chain PUFA is the % by weight of the oil. In a preferred embodiment, the % by weight of the very long chain PUFA is the % by weight of the fatty acids in an ester fraction.

In some embodiments, the oil comprises an ester fraction wherein at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% by weight of the fatty acids in the ester fraction is arachidonic acid (ARA) and the amount of ARA in the ester fraction is at least about 65%, at least about 70%, at least about 75%, at least about 80%, at least about 85%, at least about 90% by weight of the total omega-6 fatty acids in the ester fraction. In some embodiments, at least about 8%, at least about 10%, at least about 15%, at least about 20%, at least about 35%, at least about 40% by weight of the fatty acids in the ester fraction is LA. In some embodiments, the amount of LA in the ester fraction is at least about 2%, at least about 3%, at least about 4%, at least about 5%, at least about 10%, at least about 15%, at least about 20%, at least about 25% by weight of the total omega-6 fatty acids in the ester fraction.

In some embodiments, the oil comprises an ester fraction of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% by weight of the oil. In some embodiments, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99% by weight of the fatty acids in the ester fraction is ARA. In some embodiments, from about 0.5% to about 5%, about 1% to about 5%, or about 3% to about 4% by weight of the fatty acids in the ester fraction is LA.

In some embodiments, at least about 80%, at least about 85%, at least about 90%, at least about 91%, at least about 92%, at least about 93%, at least about 94%, at least about 95%, at least about 96%, at least about 97%, at least about 98% or at least about 99% by weight of the fatty acids in the ester fraction is ARA and LA.

In some embodiments, the ARA content of the fatty acids in the ester fraction is at least about 80%, at least about 85%, at least about 90%, at least about 91%, at least about 92%, at least about 93%, at least about 94%, at least about 95%, at least about 96%, at least about 97%, at least about 98% or at least about 99% by weight of the amount of ARA and LA content of the fatty acids in the ester fraction.

In some embodiments, the LA content of the fatty acids in the ester fraction is from about 0.5% to about 5%, about 1% to about 5%, or about 3% to about 4% of the ARA and LA content of the fatty acids in the ester fraction.

In a preferred embodiment, the ester fraction is an ethyl ester.

In some embodiments, the total isomer value of the oil is less than 5%, less than 4.5%, less than 4%, less than 3.5%, less than 3%, less than 2.5%, less than 2%, less than 1.5%, less than 1%, less than 0.5%, less than 0.1%, or 0%.

In some embodiments, the ARA isomer value of the oil is less than 5%, less than 4.5%, less than 4%, less than 3.5%, less than 3%, less than 2.5%, less than 2%, less than 1.5%, less than 1%, less than 0.5%, less than 0.1%, or 0%.

In some embodiments, the amount of ARA in the oil per gram of oil is from about 100 mg to about 300 mg, about 100 mg to about 600 mg, about 100 mg to about 800 mg, about 100 mg to about 900 mg, about 100 mg to about 950-mg, about 800 to about 950 mg, or 0 to about 100 mg.

Food, Supplement, and/or Pharmaceutical Compositions

In some embodiments, the present invention is a food, supplement, or pharmaceutical composition comprising an oil of the invention. The pharmaceutical composition can contain a pharmaceutically acceptable carrier.

In some embodiments, the composition is a food product. A food product is any food for non-human animal or human consumption, and includes both solid and liquid compositions. A food product can be an additive to animal or human foods. Foods include, but are not limited to, common foods; liquid products, including milks, beverages, therapeutic drinks, and nutritional drinks; functional foods; supplements; nutraceuticals; infant formulas, including formulas for pre-mature infants; foods for pregnant or nursing women; foods for adults; geriatric foods; and animal foods.

In some embodiments, the composition is an animal feed. An “animal” includes non-human organisms belonging to the kingdom Animalia, and includes, without limitation, aquatic animals, and terrestrial animals. The term “animal feed” or “animal food” refers to any food intended for non-human animals, whether for fish; commercial fish; ornamental fish; fish larvae; bivalves; mollusks; crustaceans; shellfish; shrimp; larval shrimp; artemia; rotifers; brine shrimp; filter feeders; amphibians; reptiles; mammals; domestic animals; farm animals; zoo animals; sport animals; breeding stock; racing animals; show animals; heirloom animals; rare or endangered animals; companion animals; pet animals such as dogs, cats, guinea pigs, rabbits, rats, mice, or horses; primates such as monkeys (e.g., cebus, rhesus, African green, patas, cynomolgus, and cercopithecus), apes, orangutans, baboons, gibbons, and chimpanzees; canids such as dogs and wolves; felids such as cats, lions, and tigers; equids such as horses, donkeys, and zebras; food animals such as cows, cattle, pigs, and sheep; ungulates such as deer and giraffes; or rodents such as mice, rats, hamsters and guinea pigs; and so on. An animal feed includes, but is not limited to, an aquaculture feed, a domestic animal feed including pet feed, a zoological animal feed, a work animal feed, a livestock feed, and combinations thereof.

In some embodiments, the composition is a feed or feed supplement for any animal whose meat or products are consumed by humans, such as any animal from which meat, eggs, or milk is derived for human consumption. When fed to such animals, nutrients such as LC-PUFAs can be incorporated into the flesh, milk, eggs, or other products of such animals to increase their content of these nutrients

### EXAMPLES

The following examples are set forth below to illustrate the methods and results according to the disclosed subject matter. These examples are not intended to be inclusive of all aspects of the subject matter disclosed herein, but rather to illustrate representative methods and results. These examples are not intended to exclude equivalents and variations that are apparent to one skilled in the art.

Analytical Methods:

Determination of fatty acid compositions was performed using gas chromatograph with a flame ionization detector (GD/FID) according to European Pharmacopoeia method 2.04.29.

### Example 1

Production of an ARA Ethyl Ester Concentrate

Step 1. Transesterification

Stage 1.

To a 2 L rounded-bottom flask containing 500 g of an ARA-containing oil were added 24 g of a 21% (weight basis) ethanolic solution of sodium ethoxide (Sigma) and 131 g of anhydrous ethanol. The mixture was heated to 75° C. for one hour while stirring under N² atmosphere using a reflux condenser to keep the ethanol from leaving the reaction mixture. The reaction mixture was removed from the heat and allowed to cool to about 40° C., and then it was transferred to a 2 L separatory funnel where the bottom glycerol layer was drained.

Stage 2.

The top oil layer was transferred to a clean 2 L rounded-bottom flask, and charged with an additional 2.4 g of the ethanolic solution of sodium ethoxide and 13.1 g of anhydrous ethanol. The mixture was again heated to 75° C. for one hour under N² atmosphere and a reflux condenser.

After cooling down to about 40° C., the reaction mixture was evaporated under vacuum to remove the residual ethanol. The residue was transferred to a 2 L separatory funnel, and washed with a citric acid solution (1% w/w) until the pH of the aqueous washing portions was no longer basic. The neutral oil was washed with 1.5 L of distilled water in three equal 500 mL portions, and then it was dried under vacuum at 70° C. for 2 h to yield oil containing 53.88% ARA by area, with a 95% yield.

Step 2. Short Path Distillation

The ARA ethyl esters prepared in step 1 was purified by short path distillation. The short path distillation unit was assembled according to standard protocols. The working temperature in the heating oil was set to 130° C., while the temperature in the heating unit controlling the inner condenser was set to 50° C. When the pressure in the system equilibrated to 100 mTorr, approximately 475 g of the oil was distilled in the short path unit at a flow rate of 500 g per hour. About 445 g of a clear, light-yellow distillate was obtained for further purification containing 44.56% ARA by area, with a 93% yield. The undistilled portion (residue) was discarded.

Step 3. Urea Complexation

The distilled ARA ethyl esters prepared in step 2 was concentrated by urea complexation. In a 500 mL round bottomed-flask, 50 g urea and 75 g 95% v/v aqueous ethanol were added. The mixture was placed in a heating mantle and brought to reflux while stirring in the presence of a reflux condenser. When the solution looked clear, the oil was added to the urea/aqueous ethanol mixture. The flask was removed from the heat and the mixture allowed to air cool overnight while stirring. The next day, the mixture was vacuum filtered and the liquid filtrate was evaporated under vacuum to remove the residual ethanol. The dry residue was transferred to a 500 mL separatory funnel and washed three times with three equal 100 mL portions of distilled water pre-heated to 60° C. The top oil layer from the last water wash was dried under vacuum at 70° C. for 2 h to yield an oil containing 75.2% ARA by area, with a 52.4% yield.

Step 4. Reverse Phase Chromatography

The oil prepared in step 3 was further concentrated by reverse phase chromatography. The flowing parameters were employed:

Column: Internal diameter 3.8 cm
Effective length: 27 cm
Volume: 313 mL

Silica matrix: Silicycle 60A. C-18 silica gel, carbon loading 17%, 40-63 um particle size.

A slurry of 206 g of silica gel in 500 mL of an eluent consisting of 92% methanol and 8% water in a weight basis was prepared and used to pack the column. When the column was packed, about 1 L of the eluent alone was passed through the column to equilibrate it. About 8.0 g of the ARA concentrate prepared in step 3 was loaded in neat form onto the silica gel. The column was then eluted in isocratic mode with 92/8 methanol/water mixture. The first 1.5 L of eluent (about 4.8 column volumes) passed through the column was discarded as it did not contain any oil. The remaining eluent was collected in 15 mL fractions. A total of 86 fractions were collected, for an extra 1290 mL (4.1 column volumes). Aliquots from these fractions were subjected to fatty acid profile analyses by GC. Fractions containing ARA in 89% area or higher were combined and dried under vacuum to yield an oil containing 89.34% ARA by area, with 55% yield.

Step 5. Urea Complexation

The oil prepared in step 4 was further concentrated by a second urea complexation step. In a 250 mL round bottomed-flask, 9.8 g urea and 24.5 g 95% v/v aqueous ethanol were added. The mixture was placed in a heating mantle and heated while stirring in the presence of a reflux condenser. When most of the urea had dissolved, the room temperature oil was added to the urea/aqueous ethanol mixture. The flask was removed from the heat and the mixture allowed to air cool overnight while stirring. The following day, the mixture was vacuum filtered and the liquid filtrate was evaporated under vacuum to remove the residual ethanol. The dry residue was transferred to a 125 mL separatory funnel and washed three times with three equal 50 mL portions of distilled water pre-heated to 60° C. The top oil layer from the last water wash was dried under vacuum at 70° C. for 2 h to yield an oil containing 97.35% ARA, with 51.1% yield.

Step 6. Solid Phase Extraction

The oil prepared in step 5 was further concentrated by solid phase extraction using a silica SPE cartridge. About 4.5 g of the oil material was dissolved in 10 mL of a 90/10 v/v mixture of hexanes/ethyl acetate. The solution was loaded in a Hyper Sep SI silica cartridge, which was previously equilibrated with the 90/10 hexane/ethyl acetate solvent mixture. The oil was eluted from the silica cartridge using 200 mL of the solvent mixture. The solvent was evaporated under vacuum to yield an oil containing 90.1% ARA by weight (97.3% ARA by area), with 82.2% yield.

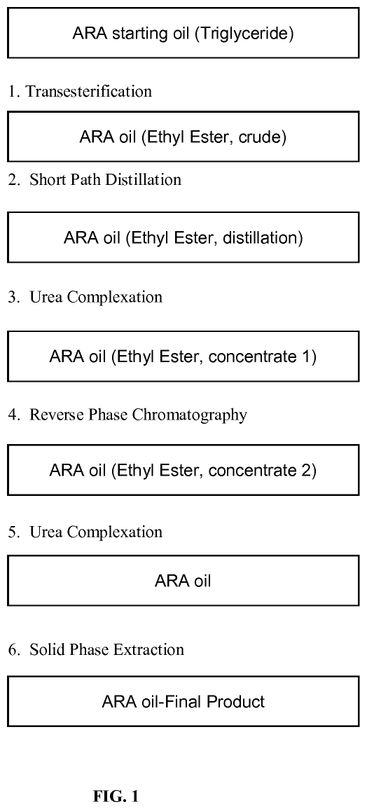

A diagram of this process is provided in FIG. 1.

### Example 2

### Comparative Example

In this example, the process performed was identical to that described in Example 1, except that the distillation step (step 2) and the solid phase extraction step (step 6) were omitted.

The final resulting oil obtained from this process contained 74.0% ARA by weight (96.9% ARA by area), with 52.0% yield.

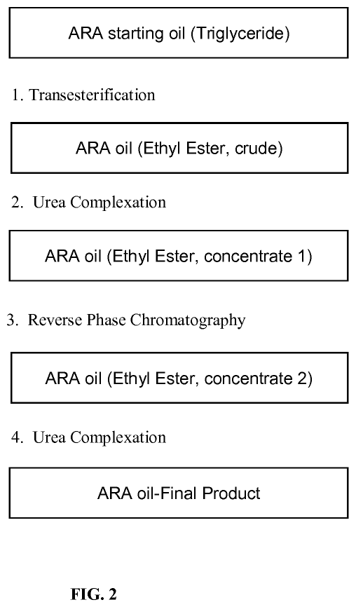

A diagram of the comparative process is provided in FIG. 2.

All references cited in this specification are herein incorporated by reference as though each reference was specifically and individually indicated to be incorporated by reference. The citation of any reference is for its disclosure prior to the filing date and should not be construed as an admission that the present disclosure is not entitled to antedate such reference by virtue of prior invention.

It will be understood that each of the elements described above, or two or more together may also find a useful application in other types of methods differing from the type described above. Without further analysis, the foregoing will so fully reveal the gist of the present disclosure that others can, by applying current knowledge, readily adapt it for various applications without omitting features that, from the standpoint of prior art, fairly constitute essential characteristics of the generic or specific aspects of this disclosure set forth in the appended claims. The foregoing embodiments are presented by way of example only; the scope of the present disclosure is to be limited only by the following claims.

What is claimed is:

1. A process for separation and concentration of an oil comprising long-chain polyunsaturated fatty acids (LC-PUFAs), the process comprising:
a) transesterification of a starting oil into its ethyl ester form;
b) distillation of the ethyl ester oil;
c) urea complexation of the distilled ethyl ester oil;
d) subjecting the oil obtained from step c) to a further concentration step;
e) urea complexation of the oil obtained from step d); and
f) filtration of the oil obtained from step e) to obtain a resulting oil,
wherein the resulting oil is enriched in at least one LC-PUFA.

2. The process according to claim 1, wherein the concentration step in step d) comprises chromatography, distillation, urea complexation, and combinations thereof.

3. The process according to claim 1 or claim 2, wherein the wt. % of the at least one LC-PUFA is increased in the resulting oil by at least about 20 wt. % as compared to the wt. % in the starting oil.

4. The process according to any of claims 1-3, wherein the wt. % of the at least one LC-PUFA is increased in the resulting oil by at least about 30 wt. % as compared to the wt. % in the starting oil.

5. The process according to any of claims 1-4, wherein the resulting oil comprises at least about 70% of the at least one LC-PUFA.

6. The process according to any of claims 1-5, wherein the resulting oil comprises at least about 80% of the at least one LC-PUFA.

7. The process according to any of claims 1-6, wherein the resulting oil comprises at least about 90% of the at least one LC-PUFA.

8. The process according to any of claims 1-7, wherein the at least one LC-PUFA is arachidonic acid (ARA).

9. The process according to any of claims 1-8, wherein the distillation step comprises fractional distillation, short path distillation, falling-film evaporation, wiped-film evaporator, or combinations thereof.

10. The process according to any of claims 1-9, wherein the distillation step comprises short path distillation.

11. The process according to any of claims 1-10, wherein the distillation step comprises fractional distillation.

12. The process according to any of claims 1-11, the starting oil is a microbial or marine oil.

13. The process according to claim 12, wherein the starting oil is a microbial oil.

14. The process according to claim 13, wherein the starting oil is produced by a microorganism, wherein the microorganism is selected from the group comprising microalgae, bacteria, fungi, and protists.

15. The process according to claim 14, wherein the microorganism is a fungus.

16. The process according to claim 15, wherein the microorganism is of the genus Mortierella.

17. The process according to claim 16, wherein the microorganism is of the species Mortierella alpina.

18. The process according to any of claims 1-17, wherein the resulting oil is transesterified back to convert at least part of the ester fraction in the oil to a triglyceride fraction.

19. An oil produced by the process according to any of claims 1-17.

20. The microbial oil of claim 19 comprising at least about 70% by weight ARA.

21. The microbial oil of claim 19 comprising at least about 80% by weight ARA.

22. The microbial oil of claim 19 comprising at least about 90% by weight ARA.

23. The microbial oil of any of claims 19-21, wherein the ARA is an ethyl ester.

24. An oil produced by the process according to any of claims 1-18.

25. The microbial oil of claim 24 comprising at least about 70% by weight ARA.

26. The microbial oil of claim 24 comprising at least about 80% by weight ARA.

27. The microbial oil of claim 24 comprising at least about 90% by weight ARA.

28. The microbial oil of any of claims 24-27, wherein the ARA is a triglyceride.

29. A microbial oil comprising at least about 70% by weight ARA.

30. The microbial oil of claim 29, comprising at least about 80% ARA.

31. The microbial oil of claim 29, comprising at least about 90% ARA.

32. The microbial oil of any of claims 29-31, wherein the ARA is an ethyl ester.

33. The microbial oil of any of claims 29-31, wherein the ARA is a triglyceride.

34. The microbial oil of any of claims 29-33, wherein the microbial oil is derived from a microorganism, wherein the microorganism is selected from the group comprising microalgae, bacteria, fungi, and protists.

35. The microbial oil of claim 34, wherein the microorganism is a fungus.

36. The microbial oil of claim 35, wherein the microorganism is of the genus Mortierella.

37. The microbial oil of claim 36, wherein the microorganism is of the species Mortierella alpina.

38. A food product, cosmetic or pharmaceutical composition for a non-human or human, comprising the oil of any of claims 19-37.

39. The food product of claim 38, wherein the food product is a milk, a beverage, a therapeutic drink, a nutritional drink, or a combination thereof.

40. The food product of claim 38 or 39, wherein the food product is an infant formula.

41. The food product of claim 38 or 39, wherein the food product is a dietary supplement.
